(19) United States
(12) Patent Application Publication
(10) Pub. No.: US 2023/0128051 A1
(43) Pub. Date: Apr. 27, 2023

(54) SYSTEM FOR SIMULTANEOUSLY ASSESSING PSYCHOLOGICAL SAFETY IN REAL TIME AND ASSOCIATED METHODS

(71) Applicant: Immersion Neuroscience, Inc., Henderson, NV (US)

(72) Inventors: Jorge A. Barraza, Claremont, CA (US); Paul J. Zak, Loma Linda, CA (US)

(73) Assignee: Immersion Neuroscience, Inc., Henderson, NV (US)

(21) Appl. No.: 17/974,609

(22) Filed: Oct. 27, 2022

Related U.S. Application Data
(60) Provisional application No. 63/272,304, filed on Oct. 27, 2021.

(51) Int. Cl. A61B 5/16; G16H 50/30; A61B 5/024; A61B 5/00

(52) U.S. Cl. CPC A61B 5/16; G16H 50/30; A61B 5/024; A61B 5/0022; A61B 2503/12

(57) ABSTRACT

A neurophysiologic assessment system for quantitatively assessing psychological safety levels of one or more experience participants within an experience and predicting participant behavior during and after the experience is described. The system includes an ingestion data hub for receiving heart rhythm data collected from the participant during the experience and processing the heart rhythm data to provide clean data. The system also includes a neuroscience processing unit for receiving and analyzing the clean data over a specific time period at predetermined intervals to generate primary metrics. The system further includes a behavior analysis unit for receiving and analyzing the clean data and the primary metrics to generate secondary metrics, and a workflow management unit for controlling the ingestion data hub, the neuroscience processing unit, and the behavior analysis unit. An associated method of using the psychological safety assessment system is also disclosed.

### CROSS-REFERENCE TO RELATED APPLICATIONS

The present application claims priority from U.S. Provisional Patent Application No. 63/272,304, filed Oct. 27, 2021, and entitled “System for Simultaneously Assessing Psychological Safety in Real Time and Associated Methods.” This application also generally relates to U.S. patent application Ser. No. 17/874,114, filed Jul. 26, 2022 and entitled “Immersion Assessment System and Associated Methods,” which in turn claims priority to U.S. Provisional Patent Application No. 63/227,544, filed Jul. 30, 2021 and entitled “Immersion Assessment System and Associated Methods.” All of the above patent references are incorporated herein by reference in their entirety.

### FIELD OF THE INVENTION

The present invention relates to the assessment of psychological safety and, more specifically, the systems and methods for performing quantitative neurophysiologic assessments in real time based on simultaneous measurement of physiologic data from one or more people.

### BACKGROUND OF THE INVENTION

Knowing what people's brains value is imperative for creating a transformative experience and has led to the proliferation of methods for assessing engagement using surveys, tests, and biometric measurements such as eye tracking, automated facial coding, electroencephalogram (EEG), Galvanic Skin Response (GSR), implicit reaction time, and, more recently, subtle changes in heart rhythms. Today, companies use such methodologies, sometimes referred to as neuromarketing, consumer neuroscience, or applied neuroscience, in fields such as advertising, marketing, training, and entertainment.

Rigorous neuroscience research in the past several decades has established a relationship between what a person is experiencing and the corresponding neurochemicals produced by that person's brain. In particular, the secretion of neurochemicals oxytocin and dopamine have been established as key signals showing that the brain values an experience. For instance, researchers have found connections between the presence of oxytocin and social behaviors such as trustworthiness, generosity, charitable giving (See, for example, 1) Zak, et al., “Oxytocin increases generosity in humans,” PLoS One 2(11): el128, 2007; 2) Zak, et al., “The neurobiology of trust,” Ann. N.Y. Acad. Sci 1032, pp. 224-227, 2004; 3) Barraza, et al., “Empathy toward strangers triggers oxytocin release and subsequent generosity,” Values, Empathy, and Fairness across Social Barriers, Ann. N.Y. Acad. Sci, 1167, pp. 182-189, 2009; 4) Barraza, et al., “Oxytocin infusion increases charitable donations regardless of monetary resources,” Hormones and Behavior, 60, pp. 148-151, 2011; 5) Lin, et al., “Oxytocin increases the influence of public service advertisements,” PLoS One 8(2): e56934, 2013); 6) Alexander, et al., “Preliminary evidence of the neurophysiologic effects of online coupons: Changes in oxytocin, stress, and mood,” Psychology & Marketing, 32(9), pp. 977-986, 2015; and 7) Zak, et al., “The neurobiology of collective action,” Frontiers in Neuroscience, 7(211), pp. 1-9, 2013).

Physiologically, the presence of oxytocin has been shown to correspondingly modulate the heart's rhythms in measurable ways (See, for example, 1) Porges, “The polyvagal theory: phylogenetic substrates of a social nervous system,” International Journal of Psychophysiology, 42(2001), pp. 123-146, 2001; 2) Thayer, et al., “Claude Bernard and the heart-brain connection: Further elaboration of a model of neurovisceral integration,” Neuroscience and Biobehavioral Reviews, 33(2009), pp. 81-88, 2009; 3) Kemp, et al., “Oxytocin increases heart rate variability in humans at rest: Implications for social approach-related motivation and capacity for social engagement,” PLoS One 7(8): 344014, 2012; 4) Norman, et al., “Oxytocin increases autonomic cardiac control: Moderation by loneliness,” Biological Psychology 86(2011), pp. 174-180, 2011; 5) Barraza, et al., “The heart of the story: Peripheral physiology during narrative exposure predicts charitable giving,” Biological Psychology 105(2015), pp. 138-143, 2015; 6) Jurek, et al., “The oxytocin receptor: From intracellular signaling to behavior,” Phsiol. Rev., 98, pp. 1806-1908, 2018; and 7) Gutkowska, et al., “Oxytocin revisited: Its role in cardiovascular regulation,” Journal of Neuroendocrinology, 24, pp. 599-608, 2012). In addition, the binding of dopamine to the prefrontal cortex is associated with the release of adrenocorticotropic hormone (ACTH) in a person's blood stream, which in turn produces changes in the person's heart rhythm (Pivonello, R., Ferone, D., Lombardi, G., Colao, A., Lamberts, S. W., & Hofland, L. J. (2007). Novel insights in dopamine receptor physiology. European journal of endocrinology, 156(1), S13.). Consequently, by monitoring subtle changes in heart rhythms, the brain's neurochemical response to an experience can be inferred such that heart rhythm data, such as collected using a photoplethysmogram (PPG), can be used to assess the person's reaction to an experience.

For instance, if a person is emotionally resonating with an experience, e.g., watching a movie or a commercial, sitting in a class, or working with a team, that person's brain typically releases oxytocin both into the brain and via the pituitary gland into the bloodstream. As oxytocin is simultaneously released into the brain and the bloodstream, a change in the oxytocin level in the blood reflects release of oxytocin in the brain. In the bloodstream, oxytocin binds to the vagus nerve and heart, thereby subtly changing the heart's rhythms (Norman et al., 2011, cited above). Thus, measurement of changes in heart rhythms can be used to infer the person's engagement with an experience at a particular moment in time.

An indicator of such a state of engagement is “immersion.” Immersion is defined as a biological state of attention and emotional resonance in the brain, as measurable by changes in the balance of neurochemicals in the brain and in the blood stream and their neuroelectrical analogs. Due to the effects of these changes on the peripheral nervous system, a person's level of immersion can also be inferred by monitoring subtle changes in the person's heart rhythms, as established in scientific research cited above. For instance, analysis of immersion has shown to predict what people will do and remember after an experience with over 80% accuracy.

Beyond immersion, the concept of psychological safety as an indicator of collaboration effectiveness has been extensively explored in the past 20+ years. Pioneered in the 1990s by Harvard University researcher Amy Edmondson, measurement of psychological safety in real life contexts is limited to self-reporting and surveys.

Unlike the psychological definition provided by Edmondson, psychological safety can be defined as a brain state that signals psychological comfort in social environments. High levels of psychological safety are considered to be an indication that a person feels relaxed, comfortable and ready to interact with others.

In the present disclosure, psychological safety is defined as a specific and measurable neurological state of readiness to engage in the social environment. This state encourages risk-taking, creativity, a sense of belonging, and a capacity to learn. Others have defined it as a subjective “psychological” belief that a person safe in a group and can take risks in a social environment (see, for example, Edmondson, “Psychological Safety and Learning Behavior in Work Teams,” Administrative Science Quarterly, Vol. 44, No. 2 (June 1999), pp. 350-383).

For instance, within a work environment, organizations are more likely to innovate quickly, unlock the benefits of diversity, and adapt well to change when employees feel comfortable asking for help, sharing suggestions with leadership, or challenging the status quo without fear of negative social consequences. Successfully creating an agile organizational structure that empowers teams to tackle problems quickly by operating outside of bureaucratic or siloed structures requires a strong degree of psychological safety. Research has shown that teams that possess two key aspects of their culture—interpersonal trust and psychological safety—are much more likely to be effective and reach performance objectives than are teams without these characteristics (Nowack, K. & Zak, P. J. (2021). Sustain high performance with psychological safety. American Society for Training & Development. February ISBN:9781952157776).

Unlike engagement, which has been measured using a variety of in-lab and in situ measurement techniques, psychological safety analyses are mostly based on self-reported measurement mechanisms such as surveys. Therefore, existing assessment techniques for psychological safety are generally considered subjective and may be influenced by a variety of factors such as the wording of the survey questions, the timing of the survey administration, and environmental factors like concerns for their reputation if they were to respond to the survey in a negative manner.

The aforementioned and other existing methods provide a quantitative assessment of psychological safety which solely rely on self-report (e.g., surveys), failing to utilize neuroscientific approaches. Accordingly, a system and method for accurately assessing psychological safety for one or more participants in a given experience utilizing a neuroscience approach would be desirable.

### SUMMARY OF THE INVENTION

In accordance with the embodiments described herein, there is described a neurophysiologic system for assessing psychological safety levels of one or more experience participants based on heart rhythm data collected from the one or more people during a specified time frame. The system includes an ingestion data hub for processing the heart rhythm data to provide clean data, and a neuroscience processing unit for analyzing the clean data over a specific time period at predetermined intervals and providing analysis results including primary metrics. The system further includes a behavior analysis unit for further analyzing the clean data and the analysis results to provide secondary metrics, and a workflow management unit for controlling the ingestion data hub, the neuroscience processing unit, and the behavior analysis unit.

In accordance with a further embodiment, the neurophysiologic assessment system includes a content control unit, interfaced with at least the neuroscience processing unit, for presenting the experience to the one or more experience participants and correlating the analysis results with specific parameters and timing associated with the experience.

In accordance with an embodiment, a neurophysiologic assessment system for quantitatively assessing psychological safety levels of one or more experience participants within an experience and predicting participant behavior during and after the experience is described. The system includes an ingestion data hub for receiving heart rhythm data collected from the participant during the experience and processing the heart rhythm data to provide clean data. The system also includes a neuroscience processing unit for receiving and analyzing the clean data over a specific time period at predetermined intervals to generate primary metrics. The system further includes a behavior analysis unit for receiving and analyzing the clean data and the primary metrics to generate secondary metrics, and a workflow management unit for controlling the ingestion data hub, the neuroscience processing unit, and the behavior analysis unit.

In accordance with a further embodiment, associated methods of using the neurophysiologic assessment system herein described is also disclosed.

In accordance with another embodiment, a method for assessing psychological safety levels of one or more participants includes collecting heart rhythm data from each one of the one or more participants, cleaning the heart rhythm data to produce clean data, analyzing the clean data over a specific time period at predetermined intervals, and generating analysis results including primary metrics.

In accordance with a further embodiment, a method for quantitatively assessing psychological safety levels of a participant within an experience is disclosed. The method includes collecting heart rhythm data from the participant during the experience, cleaning the heart rhythm data to produce clean data, analyzing the clean data over a specific time period at predetermined intervals, and generating analysis results including primary metrics.

In another embodiment, the method further includes further analyzing the clean data and the analysis results to generate secondary metrics. In certain embodiments, the secondary metrics include at least one of an analysis of the primary metrics over a duration of the experience, an analysis of the primary metrics during a specified interval within the experience, and a comparison to a database of norms based on the primary metrics.

In a further embodiment, the method further includes receiving heart rhythm data from a plurality of participants, wherein the secondary metrics include an aggregated analysis of the primary metrics from the plurality of participants.

### BRIEF DESCRIPTION OF THE DRAWINGS

FIG. 1 shows a block diagram of a neurophysiologic system for assessing the psychological safety level of one or more participants with presented content and experiences and predicting experience participant behavior, in accordance with an embodiment.

FIG. 2 shows a flow diagram for using a system for assessing the psychological safety level of one or more participants with presented content and experiences, in accordance with an embodiment.

### DETAILED DESCRIPTION OF THE EMBODIMENTS

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items, and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Various embodiments or portions of methods may also or alternatively be implemented partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such computer-readable medium may include any tangible non-transitory medium for storing information in a form readable by one or more computers, such as but not limited to read only memory (ROM), random access memory (RAM), magnetic disk storage media, optical storage media, flash memory, solid state drive, and the like.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Neuroscientists have long understood the role of oxytocin, along with the heart-brain connection, in promoting positive social behavior. Humans evolved biological mechanisms to allow risk in social settings to happen, in other words, to gauge when a social environment is safe to take risks. For example, Porges, et al. (see Porges, et al., 2001, cited above) and Thayer, et al. (Thayer, et al., 2009, cited above) have clarified the connection between the heart and the brain, specifically identifying the role of oxytocin and vagal tone (e.g., heart rate variability (HRV)) in regulating social safety. Oxytocin is associated with increased cardiac vagal tone, namely the activity of the vagus nerve influence heart rhythms (See, for example, Kemp et al., “Depression, Comorbid Anxiety Disorders, and Heart Rate Variability in Physically Healthy, Unmedicated Patients: Implications for Cardiovascular Risk,” PLoS ONE, 7(2) (2012); Norman et al., 2011, cited above), which is closely linked to the prefrontal-subcortical neural mechanism of self-regulatory function (Friedman, “An autonomic flexibility-neurovisceral integration model of anxiety and cardiac vagal tone,” Biol. Psychol., 74(2), pp. 185-199, 2007; Park, et al., “From the heart to the mind: cardiac vagal tone modulates top-down and bottom-up visual perception and attention to emotional stimuli,” Frontiers in Psychology, 5(278), 2014; Thayer, et al., 2009, cited above). According to the neuro-visceral integration model, cardiac vagal tone can index the functional integrity of the prefrontal-subcortical circuits (Friedman, 2007, cited above; Park, et al., 2014, cited above; Thayer, et al., 2009, cited above). Robust regulation of the heart via the vagus nerve, which can be indexed by higher resting HRV, is associated with more adaptive patterns of emotional responding and self-regulatory functioning, experiences of positive emotion, and resiliency to stress (see, for example, Friedman, 2007, cited above; Park, et al., 2014, cited above; Thayer, et al., 2009, cited above; Fabes, et al., “Regulatory control and adults' stress-related responses to daily life events,” Journal of Personality and Social Psychology, 73(5), pp. 1107-1117, 1997; DiPietro, et al., “Reactivity and developmental competence in preterm and full-term infants,” Developmental Psychology, 28(5), pp. 831-841, 1992; Oveis, et al., “Resting respiratory sinus arrythmia is associated with tonic positive emotionality,” Emotion, 9(2), 2009). The relationship may be bi-directional, with an increase in positive emotions leading to greater resting HRV (Kok, et al., “How positive emotions build physical health: Perceived positive social connections account for the upward spiral between positive emotions and vagal tone,” Psychological Science, 24, pp. 1123-1132, 2013). As such, cardiac vagal activity plays an important role in the assessment of the brain state of psychological safety as experienced by an individual.

As discussed above, psychological safety, like immersion, is affected by the neurochemical oxytocin, which can facilitate prosocial behavior even among strangers. Further, research has demonstrated the causal effect of oxytocin on trust by infusing synthetic oxytocin, showing that those given synthetic oxytocin were twice as likely to show maximal trust in experimental scenarios (Kosfeld, M., Heinrichs, M., Zak, P. J., Fischbacher, U., & Fehr, E. (2005). Oxytocin increases trust in humans. Nature, 435(7042), 673-676.). For instance, brain imaging experiments have shown that infusing people with oxytocin results in a marked reduction in fear-associated, brain-activity-enhancing psychological safety. That is, the more oxytocin your brain makes, the more you feel empathy toward others, connecting you emotionally and nudging you to invest in helping them.

In other words, the presence of oxytocin signals that a person is psychologically safe to be in a particular environment by reducing the natural wariness in a particular situation. While perceptions of capability, consistency, caring, candor, and authenticity as well as inherent factors such as culture, neurochemicals, and genetics all contribute to measures of psychological safety, measurement of the presence of oxytocin (either directly or through a secondary measurement of the physiological effects of oxytocin on the body), the level of psychological safety experienced by a person can be quantified.

Co-pending U.S. Provisional Patent Application Ser. No. 63/227,544, filed July 30, 2021, entitled “Immersion Assessment System and Associated Methods” and incorporated herein in its entirety by reference, describes a system and associated methods for assessing immersion, which is an indicator of a participant's engagement with a particular experience. In particular, the immersion assessment system described in the aforementioned provisional application enables simultaneous PPG data capture and assessment for one or more subjects, along with a variety of interfaces (e.g., mobile, web, and desktop applications) to provide feedback to stakeholders for reporting and workflow management. Included are also other sensing devices that enable obtaining heart rhythm data, such as built-in cameras on smartphones that utilize finger contact over the camera lens (see Coppetti, Brauchten, Muggler, et al., (2017). Accuracy of smartphone apps for heart rate measurement. European Journal of Preventive Cardiology. 24 (12), 1287-1293). The relevant heart rhythm data may include, for example, heart rate, heart rate variability, pulse rate variation, and other heart activity information. For instance, the immersion assessment system enables simultaneous evaluation of immersion levels of multiple participants experience synchronously or asynchronously, thus providing accurate behavioral prediction, especially by comparing the assessment results to a database of norms based on the primary metrics. This database of norms may be, for example, created from an aggregated set of data collected experimentally from a variety of test subjects across several studies (i.e., discrete observations with associated outcomes) using methods such as observation, surveys, and other assessments as prediction outcomes. In an embodiment, a database of norms may be integrated into the analytical algorithm implemented by neuroscience processing unit 130. As an example, the assessment scores may be weighted and normalized to fall within a numerical range of 1-100 according to a comparison to a database of norms, which database has been created from several behavioral studies of prediction outcomes.

It is recognized herein that, by analyzing heart rhythm data (such as PPG data) over a specific time period at predetermined intervals, psychological safety levels may be quantified without the use of subjective mechanisms such as surveys. That is, psychological safety may be assessed as a specific and measurable neurological state of readiness.

An exemplary process, in accordance with an embodiment, may include the following steps:

1. An experience participant is equipped with a heart data capture device, such as and not limited to a PPG-enabled smartwatch or fitness device.

2. The device outputs cardiac data, such as pulse, that is converted to heart rate data.

3. This heart rate data is delivered to the psychological safety assessment system, and is analyzed for changes in heart rate rhythms, along with other cardiac patterns associated with oxytocin release in the brain and binding on the vagus nerve.

4. The heart rate data is collected for a predetermined time period (e.g., 2 minutes or more) to observe sufficient variability in cardiac activity to determine the psychological safety indicator.

5. The system corrects the signal based on individual physiology and potential artifact or noise (e.g., movement, acceleration, or any other factor not typically associated with neurological sources of variability).

6. The system normalizes and derives a score of psychological safety.

7. Optionally, the psychological safety score is displayed for the user.

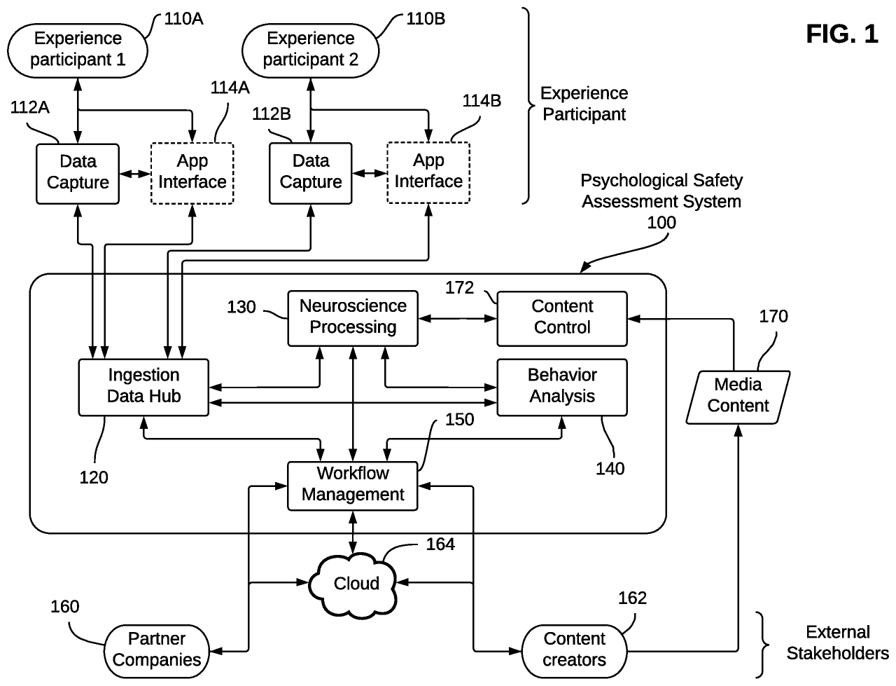

Turning now to the figures, FIG. 1 shows a block diagram of a system for assessing the level of psychological safety of one or more experience participants, or simply “participants,” in accordance with an embodiment. As shown in FIG. 1, a psychological safety assessment system 100 interfaces with a first experience participant 110A through a data capture mechanism 112A. A first experience participant 110A may be, for example, a test subject being shown a film clip, a participant at a seminar, an event attendee, or even a person going about their everyday experiences. For instance, assessment of psychological safety may be utilized in the field of human wellness, which may be expanded outside of discrete events into occurrences in everyday life. Data capture mechanism 112A is a device capable of capturing real-time heart rhythm data of experience participant 110A. As an example, data capture mechanism is a smartwatch or a fitness tracker worn by experience participant 110A to capture real-time cardiac data of experience participant 110A. Optionally, a second experience participant 110B, interfacing with a data capture mechanism 112B, may be simultaneously or serially assessed with psychological safety assessment system 100. While only two experience participants are shown in FIG. 1, psychological safety assessment system 100 may be interfaced with additional experience participants, for instance, each experience participant associated with a data capture mechanism (e.g., a smartwatch or fitness tracker worn by that experience participant).

Optionally, first experience participant 110A may interact with an application interface 114A on a mobile device or a computer. Application interface 114A may include, for example, a mobile application configured for communicating with psychological safety assessment system 100 and providing an interactive user interface for first experience participant 110A. For instance, application interface 114A may display the experience to be assessed (e.g., media content, advertisement, event recording, or live event), provide an interface for first experience participant 110A to adjust user settings, monitor data capture mechanism 112A, and/or send and receive information from psychological safety analysis system 100. Application interface 114A may also display, for example, immersion scores for first experience participant 110A, as discussed in the aforementioned co-pending U.S. provisional patent application 63/227,544. Similar functionality may be provided to second experience participant 110B via an application interface 114B, which may be the same or different (e.g., different modality or operating system) compared to application interface 114A used by first experience participant 110A.

Continuing to refer to FIG. 1, psychological safety analysis system 100 includes an ingestion data hub 120 for interfacing with first experience participant 110A and second experience participant 110B via data capture mechanism 112A and 112B and/or application interface 114A and 114B. Ingestion data hub 120 performs a variety of tasks such as pairing data from a specific experience participant with a specific event to be analyzed, clean the incoming heart rhythm data to remove illogical information (e.g., heart rate above or below specified thresholds), align incoming data from multiple experience participants with timing specific for a specific event, and calibrate the incoming data according to sensor type. Ingestion data hub 120 thus processes the incoming heart rhythm data from one or more experience participants to provide clean data.

Psychological safety analysis system 100 also includes a neuroscience processing unit 130 for performing, for example, the analysis steps outlined above. Neuroscience processing unit 130 analyzes the clean data from ingestion data hub 120 to generate analysis results as primary metrics, such as calculated psychological safety and immersion levels, by correlating received heart rhythm data with established neurochemical analyses, such as described above. Specifically, the data is processed to identify variation in the heart rhythm associated with both HRV in the high frequency range, as well as other heart rhythm patterns associated with oxytocin release in the brain. This process occurs in real time, but requires at least 2 minutes of data to begin outputting psychological safety scores for an individual or group. Neuroscience processing unit 130 presents the psychological safety scores along the experience timeline broken up into uniform time periods (e.g., 2-minute segments).

In the exemplary embodiment shown in FIG. 1, the psychological safety assessment system 100 further includes a behavior analysis unit 140. Behavior analysis unit 140 may receive the clean data from ingestion data hub 120 and analysis results from neuroscience processing unit 130 to perform further analyses such as, for instance, aggregate profiling, vertical analysis, and pattern analysis. Optionally, neuroscience processing unit 130 and/or behavior analysis unit 140 may perform additional functions such as the calculation of secondary metrics (e.g., comparison of the primary metrics with established norms), generating summary reports (e.g., norm comparison), participant breakdown (e.g., classification into categories ranging from very low psychological safety to very high psychological safety), generating annotated video of the experience with psychological safety assessment results). The primary and/or secondary metrics may optionally be sent via ingestion data hub 120 to be displayed to first and second experience participants 110A and 110B via, for example, application interface 114A and 114B, respectively.

Psychological safety assessment system 100 of FIG. 1 further includes a workflow management unit 150. Workflow management unit 150 may include, for example, interfaces with ingestion data hub 120, neuroscience processing unit 130, and/or behavior analysis unit 140 for receiving and aggregating data from each of these system components. Workflow management unit 150 may also provide an interface between psychological safety assessment system 100 with external stakeholders, or simply “stakeholders,” such as partner companies 160, who are users or clients of the psychological safety assessment system 100 or content creators 162, or provide aggregated data or analysis history to a cloud server 164. Alternatively, partner companies 160 and/or content creators 162 may interface with workflow management unit 150 via cloud server 164.

As an example, content creators 162 may include companies or personnel who produce the experience (e.g., event or media content 170) being assessed by the psychological safety assessment system 100. As another example, content creators may include content (or experience) participants who are managing the content/experience using the psychological safety assessment system 100 to organize the content/experience, invite selected experience participants to participate, and execute the measurement. Workflow management unit 150 may include a website or user interface for displaying, for instance, details related to first and second experience participants 110A and 110B and media content 170, creation and management of experiences to be assessed, as well as data and analysis results visualization in real-time during the experience and/or after the conclusion of the experience. It is noted that media content 170 may be, for instance, a video recording of a live experience, or pre-recorded content presented to one or more experience participants.

In an example, media content 170 is provided by content creators 162 to a content control unit 172 for use in presenting the experience to be assessed (e.g., audiovisual content or online event) to experience participant 110 and in correlating the analysis results of neuroscience processing unit 130 with specific event timing of media content 170. Furthermore, content control unit 172 may provide media management functions to enable secure streaming of media content 170 to specific experience participants 110, or even adjust the content provided to each experience participant 110 according to the real-time analysis results from neuroscience processing unit 130.

It is noted that, while content control unit 172 is shown in FIG. 1 as being interfaced with neuroscience processing unit 130, content control unit 172 may be additionally or alternatively interfaced with ingestion data hub 120, behavior analysis unit 140, and/or workflow management unit 150. It is further noted that, while ingestion data hub 120, neuroscience processing 130, behavior analysis unit 140, workflow management unit 150, and content control unit 172 are shown as distinct components within the psychological safety assessment system 100, two or more of these components may be combined in a single unit.

The psychological safety assessment system of the present disclosure differs from existing engagement assessment systems in that the system provides outputs based on quantitative data, namely the analysis of subtle changes in heart rhythms at predetermined intervals (e.g., every two minutes) over a specified time frame (e.g., during the first five minutes of a business meeting).

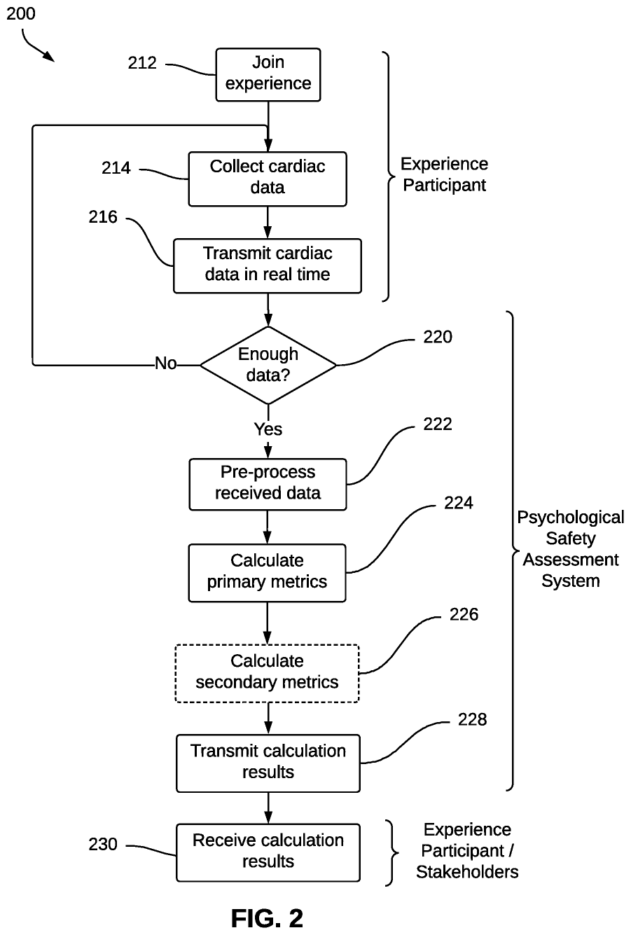

FIG. 2 shows a flow diagram for using a system for assessing the psychological safety level of an experience participant with presented content, in accordance with an embodiment. As shown in FIG. 2, in conjunction with FIG. 1, a process 200 begins when a participant (e.g., experience participant 110) opts to join an experience to be assessed (e.g., a live event or media content 170) in a step 212. Step 212 may include, for example, an opt-in agreement through an application interface or a manual signing of a waiver, in which the experience participant agrees to share physiological data (e.g., heart rhythm data) and in some cases demographic information collected during the experience. Then, in a step 214, cardiac data is collected from the participant from a device that is transmitting at least once every 5 seconds, and the collected information is transmitted to the psychological safety assessment system (e.g., psychological assessment system 100). As shown in FIG. 2, steps 212, 214, and 216 are performed at devices controlled by the participant. It is noted that multiple participants may be performing steps 212, 214, and 216 at the same time to provide input data to the neuroscience assessment system. Then the collected information from one or more experience participants are analyzed by a psychological safety assessment system in steps 220-228.

Continuing to refer to FIG. 2, the cardiac data is received at the psychological safety assessment system to determine whether sufficient data has been collected (e.g., data has been collected for a predetermined amount of time, at least two minutes) in a decision 220. If the answer to decision 220 is NO, then process 200 returns to step 214 to continue to collect cardiac data. If the answer to decision 220 is YES, then process 200 proceeds to a step 220 to pre-process the collected data. Step 222 may be performed, for example, by ingestion data hub 120. Step 222 pre-processing includes but is not limited to, identifying and correcting anomalies in the heart rhythm (e.g., out of biological ranges), and finding data gaps and inserting missing values based on expected heart rhythm patterns. Then primary metric, psychological safety, is calculated in a step 224. Step 224 may include the psychological safety analysis steps as discussed above and be performed, for example, by neuroscience processing unit 130 by correlating the input data from participants with known neurophysiologic indicators, as described above. Psychological safety calculations occur for experiences that are of sufficient length and with enough prepossessed data to identify heart rhythm patterns (e.g., 2 minutes or greater). The analysis takes a “bin” approach where the experience is broken up into predetermined bins by the system of equal duration. In real time, once enough pre-processed data is captured to complete a bin, psychological safety is derived and ready for display. Optionally, secondary metrics may be calculated in a step 226 by, for example, behavior analysis unit 140 of FIG. 1. Such secondary metrics may include aggregated analyses of primary metrics from multiple participants, analysis of the primary metrics over the duration (or smaller intervals within the duration) of the experience, and predictions of anticipated participant behavior following the experience. The calculation results of the primary and/or secondary metrics are then transmitted to participants or stakeholders in a step 228, such as via application interface 114 and/or user interface aspects of workflow management unit 150 of the psychological safety assessment system 100. Finally, the calculation received by participants or other stakeholders (e.g., partner companies 160, content creators 162, or data aggregator in cloud server 164) in a step 230.

Commonly-used PPG data collection wearables such as smartwatches and fitness trackers, or using PPG approaches using a built-in camera of a smart device, such as fingertip contact photoplethysmography (e.g., measuring finger pulse by contacting a fingertip to a built-in camera of a smart device) or non-contact photoplethysmography (e.g., using the built-in camera of a smart device to measure heart rhythm data). Heart rhythm measurement may be performed by approaches other than PPG, as long as the heart rhythm data can be collected with sufficient accuracy and resolution to enable performance of the analytic processes described below.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the claimed invention.

What is claimed is:

1. A neurophysiologic assessment system for quantitatively assessing psychological safety levels of a participant within an experience and predicting participant behavior during and after the experience, the system comprising:
an ingestion data hub for receiving heart rhythm data collected from the participant during the experience and processing the heart rhythm data so received to generate clean data;
a neuroscience processing unit for receiving and analyzing the clean data over a specific time period at predetermined intervals to generate primary metrics;
a behavior analysis unit for receiving and analyzing the clean data and the primary metrics to generate secondary metrics; and
a workflow management unit for controlling the ingestion data hub, the neuroscience processing unit, and the behavior analysis unit.

2. The neurophysiologic assessment system of claim 1, wherein the primary metrics include a calculated psychological safety level.

3. The neurophysiologic assessment system of claim 2, wherein the secondary metrics include at least one of an analysis of the primary metrics over a duration of the experience, an analysis of the primary metrics during a specified interval within the experience, and a comparison to a database of norms based on the primary metrics.

4. The neurophysiologic assessment system of claim 3,
wherein ingestion data hub is configured for receiving heart rhythm data from a plurality of participants, and
wherein the secondary metrics include an aggregated analysis of the primary metrics from the plurality of participants.

5. The neurophysiologic assessment system of claim 1, further comprising:
a content control unit interfaced with the neuroscience processing unit, the content control unit being configured for controlling the experience provided to the participant.

6. The neurophysiologic assessment system of claim 5, wherein the content control unit is further configured for providing experience parameters regarding the experience to the neuroscience processing unit, the experience parameters including at least one of event timing, key event occurrences, and event details.

7. The neurophysiologic assessment system of claim 1, wherein the neuroscience processing unit and the behavior analysis unit are configured for exchanging the primary metrics and the secondary metrics therebetween.

8. The neurophysiologic assessment system of claim 7, wherein the neuroscience processing unit, the behavior analysis unit, the ingestion data hub, and the workflow management unit are configured for exchanging the clean data, the primary metrics, and the secondary metrics therebetween.

9. The neurophysiologic assessment system of claim 8, wherein the ingestion data hub is further configured for providing at least one of the primary metrics and the secondary metrics to the participant.

10. The neurophysiologic assessment system of claim 9, wherein the ingestion data hub provides the at least one of the primary metrics and the secondary metrics to the participant upon receiving instructions from the workflow management unit.

11. The neurophysiologic assessment system of claim 1, wherein the workflow management unit is further configured for receiving instructions from a cloud server.

12. The neurophysiologic assessment system of claim 11, wherein the workflow management unit is configured for receiving, via the cloud server, instructions provided by stakeholders.

13. A method for quantitatively assessing psychological safety levels of a participant within an experience, the method comprising:
collecting heart rhythm data from the participant during the experience;
cleaning the heart rhythm data to produce clean data;
analyzing the clean data over a specific time period at predetermined intervals; and
generating analysis results including primary metrics.

14. The method of claim 13, wherein the primary metrics include a calculated psychological safety level.

15. The method of claim 14, further comprising further analyzing the clean data and the analysis results to generate secondary metrics.

16. The method of claim 15, wherein secondary metrics include at least one of an analysis of the primary metrics over a duration of the experience, an analysis of the primary metrics during a specified interval within the experience, and a comparison to a database of norms based on the primary metrics e based on the primary metrics.

17. The method of claim 16, further comprising receiving heart rhythm data from a plurality of participants, wherein the secondary metrics include an aggregated analysis of the primary metrics from the plurality of participants.

18. The method of claim 13, further comprising controlling the experience by selecting specific content presented to each one of the one or more participants as the experience.

19. The method of claim 13, wherein generating analysis results occurs in real time as the participant is presented with the experience.
